(19) United States
(12) Patent Application Publication
(10) Pub. No.: US 2005/0118245 A1
(43) Pub. Date: Jun. 2, 2005

(54) Assembled unit consisting of individually separable, transdermal, therapeutic systems

(76) Inventor: Klaus M. Wilsmann, Roetgen (DE)

Correspondence Address: Geza C. Ziegler, Jr.;PERMAN & GREEN, LLP, 425 Post Road, Fairfield, CT, 06824, US

(21) Appl. No.: 10/950,891

(22) Filed: Sep. 27, 2004

Related U.S. Application Data
(63) Continuation of application No. PCT/EP03/03004, filed on Mar. 22, 2003.

(30) Foreign Application Priority Data
Mar. 27, 2002 (DE) DE 102 13 772.2

(51) Int. Cl. A61F 13/00

(52) U.S. Cl. 424/449

(57) ABSTRACT

The invention relates to an assembled unit consisting of individually separable transdermal therapeutic systems.

The invention relates to an assembled unit consisting of individually separable, transdermal, therapeutic systems.

Transdermal, therapeutic systems for the application of active substances to be administered transdermally are known. These are technically specified plaster systems that, while being continuously monitored over a specific period, impart a therapeutic active substance to a human or animal organism, via the skin.

In said systems, the active substance may be dispersed in a matrix or be found in an active substance reservoir. Both in the matrix and in the active substance reservoir, the active substance may be used in liquid, semi-solid or solid form, or as a corresponding active substance formulation.

The known transdermal, therapeutic systems contain a specific predetermined dosage of the active substance, so as to release it in a controlled manner. Administering the individual dose of the active substance, in accordance with the patient's requirements, is problematic.

According to the prior art, the individual dose is administered in various ways.

According to the teaching of DE-U-295 11 878, a strip-shaped, transdermal, therapeutic system is proposed, from which a piece may be separated, in accordance with the patient's requirements. The separation takes place in the active substance-containing region. Amounts of active substance may be lost in this way, and even small separation imprecisions may change the dosage of the active substance. It is therefore impossible to administer the dose exactly.

The publication DE 197 33 981 discloses a transdermal, therapeutic system, the active substance-containing region of which may be partially covered in order to reduce the dosage. However, this may result in part of the active substance not being used. The contact of the active substance-containing region with the skin may also be impaired.

The publication DE 199 00 645 discloses a transdermal, therapeutic system consisting of individually separable, active substance-containing matrix portions, each of which may be applied, individually or several at once, using adhesive strips. The use of a matrix system and separated adhesive strips is, however, complicated, and does not ensure optimal contact of the adhesive substance-containing matrix portions with the skin.

The object has therefore been set of providing a transdermal, therapeutic system for the application of active substances to be administered transdermally, that is easy to use, that allows the individual dose of active substances to be administered exactly, in accordance with the patient's requirements, without losing active substance and ensuring reliable skin contact.

According to the invention, said object is achieved by providing an assembled unit consisting of at least two individually separable, transdermal, therapeutic systems, each comprising:
a) a base film that is impermeable to active substances,
b) a layer that is capable of adhesion, that at least partially covers the base film, that comprises either an active substance reservoir or an active substance-containing matrix, and that has a peripheral edge region that is free of active substances, and optionally
c) a detachable protective film that at least partially covers the layer that is capable of adhesion,
wherein the individually separable, transdermal, therapeutic systems comprise separation facilities in their adjacent edge regions that are free of active substances.

There are preferably linear perforations for separating the individually separable, transdermal, therapeutic systems from the assembled unit according to the invention.

Cutting marks on the base film and/or the cover film, which indicates the facility for separating the individually separable systems from the unit according to the invention, are also preferred. The separation facilities are, in each case, preferably arranged such that they allow each assembled, transdermal, therapeutic system to be separated completely from the unit according to the invention.

Any active substances that may be administered transdermally, such as corticosteriods, analgesics, sedatives, tranquillisers, antibiotics, anaesthetics, antiviral agents, antimicrobial agents, fungicides, vitamins, anticonvulsants, sexual hormones, nicotine, psychopharmacological agents, coronary dilators, anti-arthritic agents, anti-asthmatic agents, antidepressants, antidiabetic agents, antihistamines, anti-inflammatory agents and/or anti-migraine agents, are suitable.

The unit according to the invention or a separated system is preferably suitable for combating pain.

The individually separable, transdermal systems comprise a base film and thereupon a layer that is capable of adhesion, that is provided with an active substance-containing matrix or an active substance reservoir, and that may optionally comprise a protective film.

The layer that is capable of adhesion may consist, at least in certain sections and preferably completely, of a matrix material that contains a component that is capable of adhesion, preferably a pressure-sensitive adhesive component, and comprises an active substance-containing region. Said active substance-containing region is surrounded by a peripheral edge region that is free of active substances.

The base film that is impermeable to active substances is preferably made of a flexible, extendable, durable material with good breathing properties. A textile-type fabric is particularly preferred. The base film may be coloured, preferably skin-coloured.

The term “impermeable to active substances” means that the base film is impermeable to active substances at least in the region in which there is an active substance-containing matrix. If the base film itself is not impermeable to active substances, a barrier layer that is impermeable to active substances should be applied between the matrix layer or the active substance reservoir and the base film. Said barrier layer should preferably comprise a film-forming polymer.

The matrix material of the layer b) may be based on lipophilic or hydrophilic polymers. Hydrophilic polymer matrices may contain water, and are preferably gels. The matrix materials may be crosslinkable polymers, preferably synthetic resins, silicone rubbers, or rubbers such as styrene-isoprene-styrene block copolymers, silicones, polyacrylates, polyurethanes, polyvinyl ether, polyvinyl chloride, polyvinyl alcohols, polyvinylpyrrolidones, polyester, polypropylene and/or polysaccharide. Polyacrylates are particularly preferred.

The component capable of adhesion that is used for producing the layer capable of adhesion is preferably a skin-compatible polymer adhesive, particularly preferably a pressure-sensitive adhesive, such as hot melt.

In order to produce the layer that is capable of adhesion, the component that is capable of adhesion may be mixed with the above-mentioned matrix materials in known quantities, and in order to produce the active substance-containing matrix region, an active substance may be added. The active substance-containing matrix region is applied to the base film such that there is a peripheral edge region that is free of active substances. After the application, the matrix material may, if necessary, also be crosslinked. The active substance or substances that is/are found in the matrix may be in liquid, semi-solid or solid form, in the dispersed state, or be incorporated as a corresponding formulation, by adding conventional auxiliary materials as the active substance formulation.

Compounds that strengthen or facilitate the transdermal conveyance of the active substances may be used as conventional auxiliary materials. Said auxiliary materials may be mixed with the active substances or be found in a layer that is separate from the active substance-containing matrix layer.

In so far as the active substances to be applied transdermally are located in an active substance reservoir in the layer that is capable of adhesion, said reservoir is preferably embedded in the layer that is capable of adhesion. The above-mentioned polymer adhesives, which were listed as the component that is capable of adhesion, are suitable adhesives for said layer. The active substance reservoir preferably contains the active substance or the corresponding active substance formulation as a solution that is able to diffuse, unimpeded, through the membrane of the active substance reservoir. The above-mentioned polymers, which may also be used as the matrix material, are suitable membrane materials.

If, exceptionally, the active substance-containing matrix region of the layer b) does not contain a component that is capable of adhesion, the transdermal system may also be configured such that the component that is capable of adhesion, as the matrix component, is found only in the edge zone, which is free of active substances.

All of the materials that are used to construct the transdermal systems, but in particular those materials that come into contact with the skin, must be skin-compatible and physiologically safe.

The transdermal, therapeutic system according to the invention conventionally comprises a protective film, which may easily be removed prior to application, at least in the active substance-imparting region, and preferably over the entire layer that is capable of adhesion. Said protective film is preferably made of paper, or is a physiologically safe, plastics material film that adheres easily.

The assembled unit according to the invention, consisting of individually separable, transdermal, therapeutic systems, preferably contains the same dosage of active substance for each system, as a result of which the individual, requirement-dependent application is facilitated considerably for the patient or user. This is particularly true if the active substance requirements vary, in the event of recurring bouts of pain, for example, or if a diminishing treatment, with a decreasing dosage, is required. The application, for this purpose, of a plurality of transdermal, therapeutic systems according to the invention allows a suitable initial treatment, and the active substance dosage may easily be reduced, as appropriate, by the patient himself, during the subsequent course of the treatment, by reducing the number of systems.

The assembled unit according to the invention, consisting of individually separable, transdermal, therapeutic systems, is preferably packaged, a sterile packaging also being possible, if required.

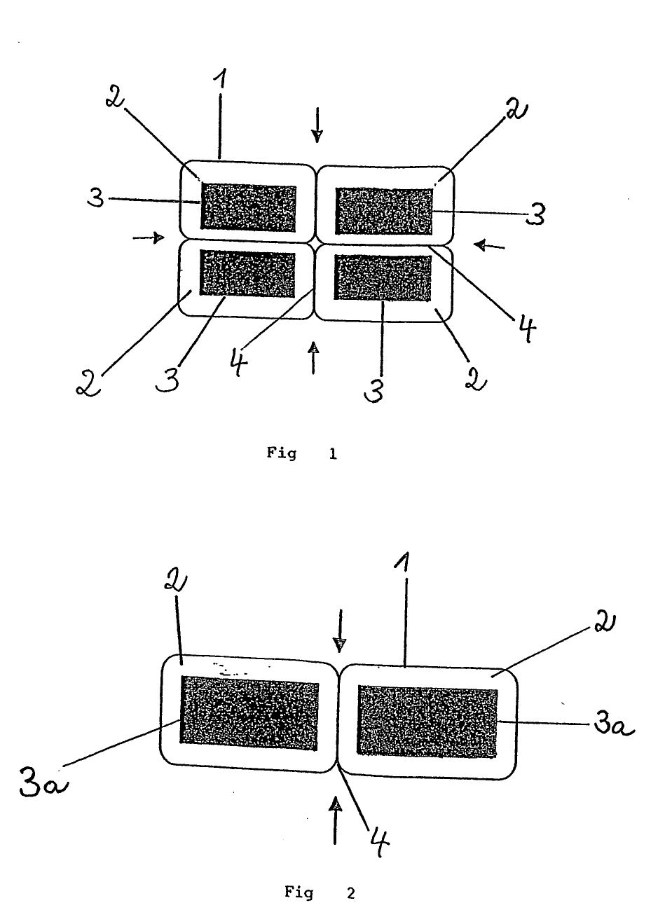

FIG. 1 shows a plan view of a unit (1) according to the invention, consisting of four individually separable, transdermal, therapeutic systems; and

FIG. 2 shows a plan view of a unit (1) according to the invention, consisting of two individually separable, transdermal, therapeutic systems.

Both in FIG. 1 and in FIG. 2, the arrows point in the direction of the separation lines (4), in this case perforations, of the respective individually separable, transdermal system of the remaining unit (1). Each individually separable system comprises an active substance-containing matrix region (3) or an active substance-containing reservoir (3a), which is surrounded by a peripheral edge region (2) that is capable of adhesion and is free of active substances. The base film and an optional protective film are not illustrated separately in FIGS. 1 and 2.

1. Assembled unit consisting of at least two individually separable, transdermal, therapeutic systems, each comprising:
a) a base film that is impermeable to active substances,
b) a layer that is capable of adhesion, that at least partially covers the base film, that comprises either an active substance reservoir or an active substance-containing matrix, and that has a peripheral edge region that is free of active substances, and optionally
c) a detachable protective film that at least partially covers the layer that is capable of adhesion,
wherein the individually separable, transdermal, therapeutic systems comprise separation facilities in their adjacent edge regions that are free of active substances.

2. Unit according to claim 1, characterised in that the separation facility is a perforation.

3. Unit according to claim 1, characterised in that the separation facility is indicated by a cuffing mark.

4. Unit according to claim 1, characterised in that the base film is made of fabric, preferably a textile-type fabric.

5. Unit according to claim 1, characterised in that at least one therapeutic active substance is dispersed in a matrix or is found in the active substance reservoir.

6. Unit according to claim 1, characterised in that the active substance in the active substance reservoir is in that form of a solution that is surrounded by a membrane that is permeable to active substances.

7. Unit according to claim 6, characterised in that each individually separable, transdermal system contains that same dosage of active substances.

8. Unit according to claim 1, characterised in that it is covered by a packaging.
